(19) United States
(12) Patent Application Publication
(10) Pub. No.: US 2011/0159477 A1
(43) Pub. Date: Jun. 30, 2011

(54) PRODUCTS AND ANALYTICAL METHOD

(76) Inventors: Kurt Nilsson, Lund (SE); Åsa Kronblad, Oxie (SE)

(21) Appl. No.: 12/997,310

(22) PCT Filed: Jun. 10, 2009
(86) PCT No.: PCT/SE2009/050706

§ 371 Date: Mar. 9, 2011

(30) Foreign Application Priority Data
Jun. 10, 2008 (SE) 0801352-6

(51) Int. Cl. C12Q 1/70; G01N 33/53

(52) U.S. Cl. 435/5; 436/501; 435/7.32; 435/7.2

(57) ABSTRACT

The present invention describes product and analysis method for analysis of a substance, a virus, a bacteria or a cell which binds to carbohydrate structures. One or more carbohydrates, or carbohydrate derivatives, is bound to spherical or non-spherical polymer beads (for example microspheres or nanospheres). Formed carbohydrate-polymer beads or carbohydrate-polymer particles, can bind to with more or less specific affinity to the substance, bacteria, virus or cell which shall be detected. The carbohydrate beads is contacted with a sample containing the substance, bacteria, virus or cell (completely or partially isolated, or not purified, in a non-diluted or diluted solution). The resulting mixture is optionally added to a gel, or a column containing a gel, through which the resulting mixture (of carbohydrate beads or carbohydrate particles bound to the substance, virus, bacteria or cell), is allowed to migrate. Detection of aggregates is made visually or using apparatus for detection.

The present invention describes product and analysis method for analysis of a substance, a virus, a bacteria or a cell which binds to carbohydrate structures.

Substances, viruses, bacteria or cells which binds to carbohydrates can be determined quantitatively and qualitatively with several methods as for example enzyme linked immunoadsorption assay (ELISA), biosensors, microarrays and flow cell techniques.

The present invention involves products and methodology which is characterised by that it involves at least more than one of the following steps:
1. One or more carbohydrates, or carbohydrate derivatives, is bound to spherical or non-spherical polymer beads (for example microspheres or nanospheres). It is formed carbohydrate-polymer beads or carbohydrate-polymer particles, which can bind to with more or less specific affinity to the substance, bacteria, virus or cell which shall be detected.
2. The carbohydrate beads is contacted with a sample containing the substance, bacteria, virus or cell (completely or partially isolated, or not purified, in a non-diluted or diluted solution).
3. The resulting mixture is added to a gel, or a column containing a gel, through which the resulting mixture (of carbohydrate beads or carbohydrate particles bound to the substance, virus, bacteria or cell), is allowed to migrate.
4. Detection.

The polymer beads which are mentioned above under 1, can consist of for example polystyrene beads, other polymer beads or copolymer beads and their chemical composition is not limiting for the invention. The polymer beads can according to the invention be magnetic (for example so called super-paramagnetic beads) or non-magnetic, for example polystyrene beads. The particles can be coloured according to the invention to facilitate detection. The size or diameter of the particles or beads, their concentration (number per volume) and density as the above parameters are chosen by the expert in the field and do not limit the scope of the invention. The size of the bead can be for example of a medium size which falls in the range 10 nm to 10 mikrometer. The exact size is determined by the exact application by the expert in the field.

Polymer beads can bind to carbohydrate or carbohydrate derivative covalently, or non-covalently.

Preferably covalent binding is applied according to the invention between polymer bead, or particle, and carbohydrate or carbohydrate derivative. Different chemical groups are used for binding between polymer particle, or bead, and carbohydrate or carbohydrate derivative, such as for example, amino group, carboxyl group via for example carbodiimide or succinimide derivatives to achieve an amide linkage between the amino group and the carboxyl group. For example can the polymer particle or bead be derivatised with an amino or a carboxyl group and the carbohydrate or carbohydrate derivative be derivatised with an carboxyl or amino group and an amide group, as a covalent linkage, can be formed between the particle, or bead, and the carbohydrate, or carbohydrate derivative, using for example EDC or NHS (N-hydroxysuccinimide) to promote amide formation. There are many other alternatives for covalent binding, which can be chosen by the expert in the field and this do not limit the scope of the invention, for example binding of amino group containing carbohydrate, to epoxid group, or to tosylat group, containing particle bead or particle. The polymer bead or particle can also for example contain covalently bound avidin or streptavidin, which can be used for non-covalent binding to the particle or bead, of biotin containing carbohydrate derivative, the reverse can also be used for non-covalent binding, i.e. biotin containing bead or particle and avidin or streptaviding containing carbohydrate derivative.

The choice of binding method, as well as the conditions for binding, such as reaction time, temperature, pH and concentration of reagents, as well as the desired quantity of bound carbohydrate or carbohydrate derivative per particle, is decided by the expert in the field and this do not limit the scope of the invention.

In step 2, the sample can be purified using for example an affinity column or affinity gel, for example containing a covalently bound carbohydrate which bind to the substance, virus, bacteria or cell to be analysed. After elution of the substance, virus, bacteria or cell, the substance, virus, bacteria or cell has been purified to a certain extent and disturbances from other compounds in the original sample, for example blood, blood serum or other biological sample, can thereby be minimized according to the invention. Concerning step 2 above, the equipment, the quantity, porosity, diameter and chemical characteristics of the affinity gel (for example agarose or derivatised agarose, or cross-linked agarose or other separation material with bound biomolecule, protein or carbohydrate) and conditions are decided by the expert in the field and these parameters are not limiting the scope of the invention.

Concerning step 3 above, to promote the migration of the particles or beads through the gel or gel column, can according to the invention be used for example centrifugation of the gel, or application of a magnetic field over the gel or gel column (in the case where magnetic beads are used according to the invention). Alternatively an electric field can be applied to promote migration. The apparatus and conditions (for example time, speed, temperature) for centrifugation as well as apparatus and conditions for application of the magnetic field or the electric field are chosen by the expert and this is not limiting the scope of the invention.

Non-limiting examples of gel which can be used are so called microtyping cards which can be used e.g. for analysis of proteins, antibodies, virus, bacteria or cells, which can bind to for example one or more of for example other proteins, carbohydrates, red blood cells or other cells, or virus. Examples hereof are the ID Microtyping cards which are sold be Diamed. These cards can be used equipped with, or not be equipped, with antibody or protein specific for the substance or for the group which the substance belongs to (for example the group IgG or the group IgM). Other gels may be chosen by the expert and do not limit the scope of the invention. Also other types of gels, for example gels used for electrophoreses can be used. The porosity, quantity, size of gel, gel beads and composition of the gel is determined by the expert in the field and do not limit the scope of the invention.

When there is a sufficient amount of the substance, virus bacteria or cell present in the sample one desire to analyse according to the invention, and where the substance, bacteria, cell or virus have a more or less pronounced specific affinity for the carbohydrate or carbohydrate derivative, or the carbohydrate derivative on the polymer bead or particle, an aggregate or agglutinate is formed which is more or less spread out over the gel. When there is a sufficient quantity of substance, virus, cell or bacteria in the sample, a more or less complete agglutinate can be formed which will not migrate through the gel and which can detected for example on the top (on the application area of the sample) of the gel. When the quantity of the substance, bacteria, virus or cell is decreased, more of the particles will not aggregate or form agglutinates, and more particles will migrate through the gel, and more particles will be detected at the bottom of the gel.

The expert is optimizing the conditions for each assay and the exact conditions for the assay do not limit the scope of the invention.

The agglutinates in the gel above can be detected visually, or with for example a microscope with or without a computer programme adapted to reading and analysing the agglutinate/aggregates over the gel, or with a scanner reading the density of particles over the gel.

Detection can also be made according to the invention without step 3 above, using a microscope supplied with a computer programme adapted to reading, analysing and calculating the quantity of aggregates and agglutinates formed in step 2 above, between carbohydrate containing polymer particles or beads, and the substance, virus, bacteria or cell to be analysed.

Non-limiting examples of carbohydrates and carbohydrate derivatives according to the present invention, is one or several of carbohydrates found in glycoproteins, glycopeptides, glycolipids, mono-, di-, tri-, tetra and higher oligosaccharides, monovalent, divalent, or multivalent containing one or more specific carbohydrate sequences, derivatives of the said saccharides for example containing O-, N-, or S-glycosides of these substances, where the aglycon comprises for example an aliphatic or aromatic part and for example a terminal amino- or carboxyl group for covalent binding to the polymer particle or bead as described above, or comprises a biotin, avidin or streptavidin molecule for noncovalent binding as described above.

Specific non-limiting examples of carbohydrate structures which can be used according to the invention, are carbohydrate structures containing the blood group determinants such as type 1, type 2, type 3 or type 4 of blood group A, B, AB, or O, mono-, di-, tri-, tetra- or higher saccharide parts thereof, the Lewis a, b, x or y substances, sialic acid containing carbohydrate structures, ganglioside structures, such as for example GM1, or parts thereof, or Galili structures, Galalfa1-4Gal-, Galalfa1-3Gal- containing structures, lactosamine containing structures, GlcNAcbeta1-3Gal, GalNAcbeta1-3Gal containing structures. One or more saccharide structures can be used bound to polymer particles. The choice of carbohydrate or carbohydrate derivative and the concentration of the carbohydrate or carbohydrate derivative is made by the expert for the specific application and do not limit the scope of the invention.

The substances to be analysed are characterised by that they can bind to the carbohydrate or carbohydrate derivative. Examples are antibodies or proteins, virus, bacteria or cell which bind to one or more of the above exemplified carbohydrate structures.

Below the use of the invention is exemplified for detection of anti-blood group A or B antibodies.

Determination of anti-A or anti-B antibodies is important with regards to for example ABO-incompatible transplantation. Inter-center variation in A/B antibody titrations is common due to a lack of standardised protocols. Different transplant centres achieve different titre levels even when the same method, erythrocytes and plasma are used.

Donor erythrocytes are frequently used as reference erythrocytes in ABO-incompatible transplantations titre measurements, as it is impossible to use the same erythrocytes world-wide.

A more reproducible, standardised method is required to achieve exact and comparable titres. The carbohydrate based particle agglutination assay according to the present invention was used to enable standardised anti-NB antibody determination as described below.

Method-2

1. Plasma (Blood group O)

2. Dilute with ID-diluent-2 (DiaMed) (LISS buffer)

3. Mix dilutions with Glycobeads-A or B,

4. Incubate for 30 min, RT (DAT) or 37° C. (IAT)

5. Method-1, steps 7-8.

METHOD-2

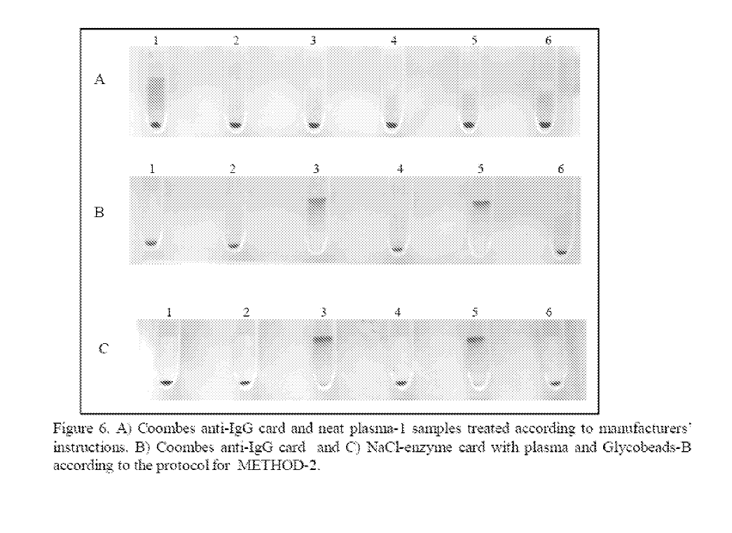

The recommended protocol for RBCs and Gelcards required optimisation in order to detect anti-B antibodies with Glycobeads-B directly in the plasma (see FIG. 6A). Neat plasma and Glycobeads did not agglutinate, see FIGS. 6B and C, lane 1,2.

100 μl neat plasma or Glycosorb-B treated plasma with addition of 25 or 50 μID-diluent-2 caused the Glycobeads-B to agglutinate in a specific manner, shown in FIGS. 6B and C, lane 3-6.

Method-1 Results

Glycobeads A

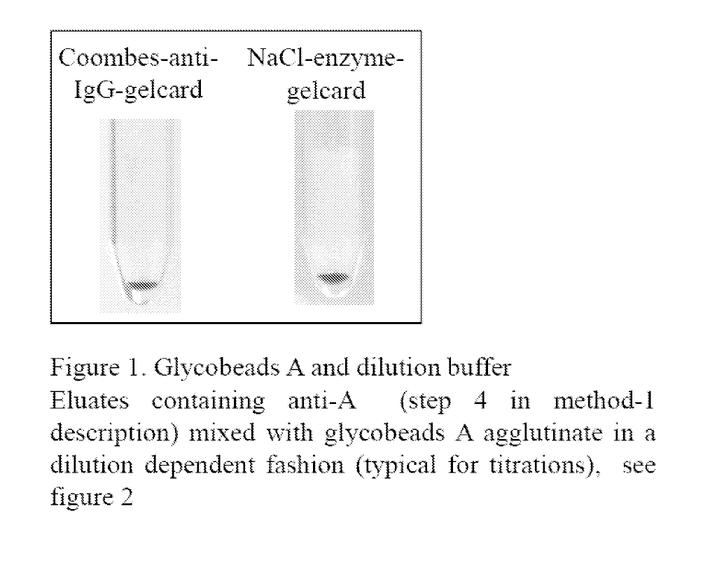

Negative control (glycobeads A mixed with dilution buffer) passed the gel after centrifugation, see FIG. 1.

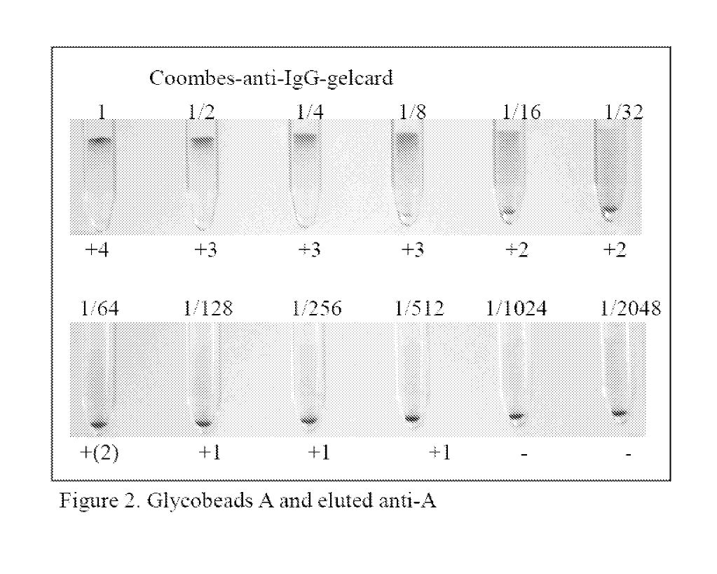

Column eluates containing anti-A antibodies (step 4 in method-1 description above) were mixed with Glycobeads-A. Agglutinates were formed in a dilution dependent fashion, typical for titrations, see FIG. 2.

Glycobeads B

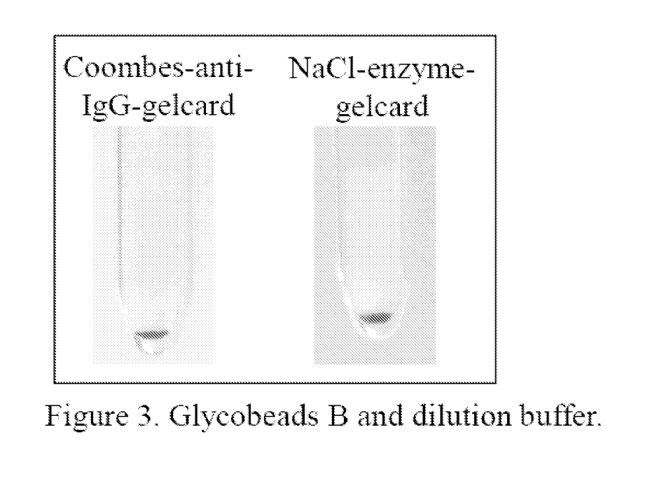

Negative control (glycobeads B mixed with dilution buffer) passed the gel after centrifugation, see FIG. 3

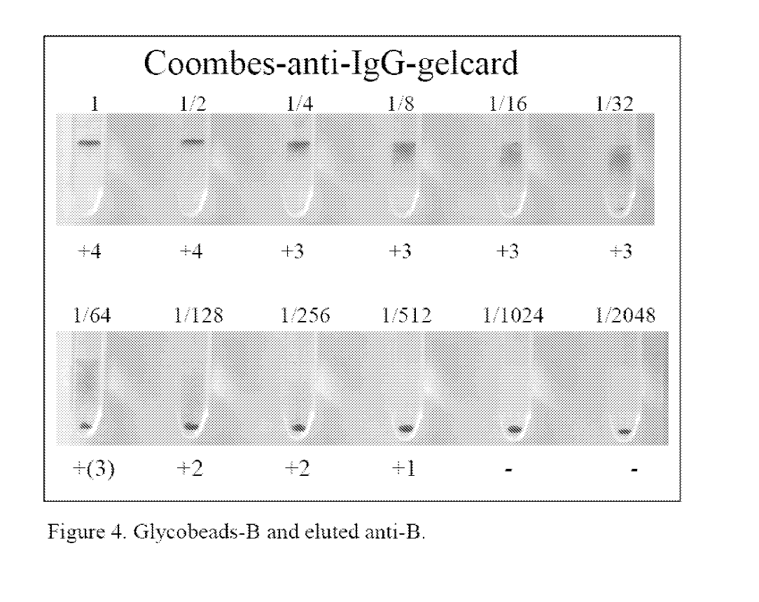

Column eluates containing anti-B antibodies (step 4 in method-1 description above) were mixed with Glycobeads-B. Agglutinates were formed in a dilution dependent fashion, typical for titrations, see FIG. 4.

Comparison With Erythrocyte Titrations

Three fresh frozen blood group O plasma samples with known anti-B titres, which had been obtained with erythrocyte titrations (shown in red) were tested with the Glycobeads-B Gelcard method (shown in blue).

Eluates were diluted in a typical titration series before mixing with the Glycobeads. The last dilution which gave a +2 reaction were said to be the titer. Results and the titers are given in table 1.

TABLE 1
Agglutination grades and obtained titres from three different blood group O plasma
samples with the erythrocyte titration method and Glycobeads-B titration method.

| Titer | | | | | | | | | | | | |
|---|---|---|---|---|---|---|---|---|---|---|---|---|
| dilution | | 1 | 2 | 4 | 8 | 16 | 32 | 64 | 128 | 256 | 512 | Titer |
| Plasma | IAT-RBC | 4 | 4 | 4 | 4 | (4) | 3 | 3 | 2 | (2) | 0 | 256 |
| 0-1 | DAT-RBC | 4 | 4 | 4 | 3 | 2 | (2) | 0 | 0 | 0 | 0 | 32 |
| | IAT-Glycobead B | 3 | 3 | 3 | 3 | 3 | 2 | 2 | 1 | 0 | 0 | 64 |
| | DAT-Glycobead B | 3 | 3 | 2 | 2 | 2 | 1 | 0 | 0 | 0 | 0 | 16 |
| Plasma | IAT-RBC | 4 | (4) | 3 | 3 | 3 | 2 | (2) | 0 | 0 | 0 | 64 |
| 0-2 | DAT-RBC | 4 | 3 | 3 | (2) | (1) | 0 | 0 | 0 | 0 | 0 | 8 |
| | IAT-Glycobead B | 3 | 3 | 3 | 2 | 2 | 2 | 2 | 2 | 0 | 0 | 128 |
| | DAT-Glycobead B | 3 | 2 | 2 | 2 | 2 | 0 | 0 | 0 | 0 | 0 | 16 |
| Plasma | IAT-RBC | 4 | 3 | 3 | 2 | 1 | 0 | 0 | 0 | 0 | 0 | 8 |
| 0-3 | DAT-RBC | 4 | 3 | 2 | 1 | 0 | 0 | 0 | 0 | 0 | 0 | 4 |
| | IAT-Glycobead B | 3 | 3 | 2 | 2 | 3 | 0 | 0 | 0 | 0 | 0 | 8 |
| | DAT-Glycobead B | 3 | 3 | 2 | 2 | 1 | 0 | 0 | 0 | 0 | 0 | 8 |

Linearity Test of Glycobead-B and Eluted Anti-B

Two different volumes (10 ml or 2.5 ml) of the same blood group O plasma were passed though the glycosorb B column and elated in 10 ml. Titrations with Glycobeads-B and eluted anti-B showed a two titer step reduction confirming that the binding between Glycoheads-B and anti-B were linear. Results are shown in table 2 for plasma O-1.

TABLE 2
Linearity test of Glycobead B binding to anti-B

| Titer | | | | | | | | | | | |
|---|---|---|---|---|---|---|---|---|---|---|---|
| dilution- | | | | | | | | | | | |
| Plasma | | | | | | | | | | | |
| O-1 | 1 | 2 | 4 | 8 | 16 | 32 | 64 | 128 | 256 | 512 | Titer |
| IAT | 3 | 3 | 3 | 3 | 3 | 2 | 2 | 1 | 0 | 0 | 64 |
| IAT ¼ | 3 | 3 | 3 | 0 | 2 | 1 | 0 | 0 | 0 | 0 | 16 |
| DAT | 3 | 3 | 2 | 2 | 2 | 1 | 0 | 0 | 0 | 0 | 16 |
| DAT ¼ | 2 | 2 | 2 | 1 | 0 | 0 | — | — | — | — | 4 |

Agglutination Grade Score And Scanning Of Glycobeads B Gel Card Assay

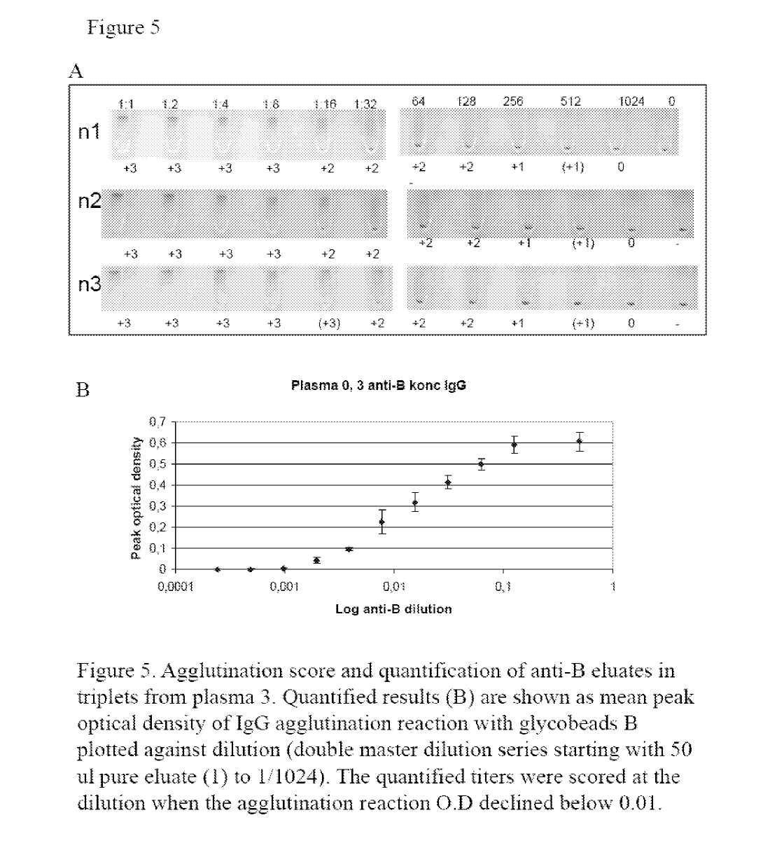

Besides macroscopical evaluation the Glycosorb B gel cards were scanned with a GS-710 calibrated densitometer (Bio-Rad Laboratories AB, Life Science, Sundbyberg, Sweden) and analysed for peak optical density with Quantity One version 4.0 software (Bio-Rad). The density determination was divided in three regions, one at the top of the gel, one middle region and one bottom region. Analysis showed that only the top part of the gel was informative and most accurate for optical density. Both front and backside of all cards were scanned and mean peak optical density was evaluated. Agglutination grade score and scanned quantified results of anti-B eluates from plasma 3 are shown in FIGS. 5A and B, respectively.

Optimisation

The results were the same regardless of whether 0.1 ml or 1 ml Glycosorb columns were used and whether the time in which the assay was performed was 45 or 90 min. The agglutination pattern with Glycobeads-B are shown in table 3 for plasma O-2 with both methods.

TABLE 3
Optimisation of elution method.

| Titer-dilution | | | | | | | | | | | | |
|---|---|---|---|---|---|---|---|---|---|---|---|---|
| Plasma O-2 | | 1 | 2 | 4 | 8 | 16 | 32 | 64 | 128 | 256 | 512 | Titer |
| 1 ml glycosorb | IAT | 3 | 3 | 3 | 2 | 2 | 2 | 2 | 2 | 0 | 0 | 128 |
| B column-90 | DAT | 3 | 2 | 2 | 2 | 2 | 0 | 0 | 0 | 0 | 0 | 16 |
| min assay | | | | | | | | | | | | |
| 1 ml glycosorb | IAT | 3 | 3 | 3 | 3 | 2 | 2 | 2 | (2) | 1 | 0 | 128 |
| B column-45 | DAT | 4 | 3 | 3 | 2 | (2) | 1 | 0 | 0 | 0 | 0 | 16 |
| min assay | | | | | | | | | | | | |

Titration Of Plasma From A Glycosorb A Treated Patient

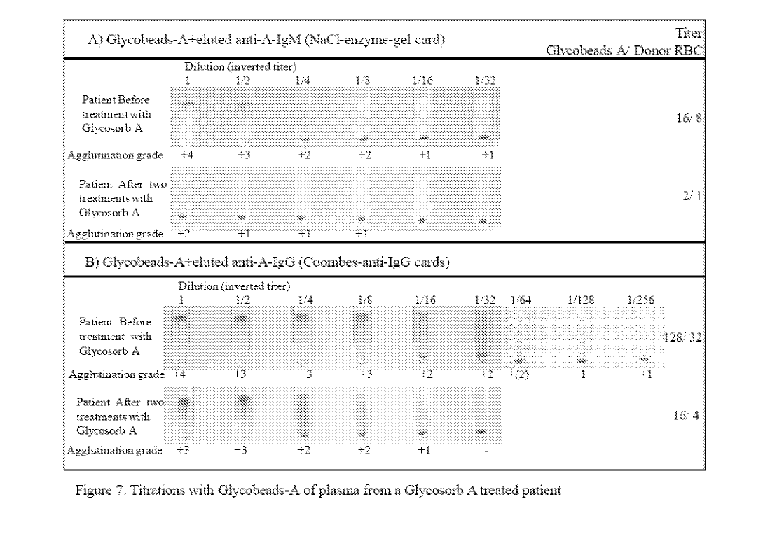

The optimised protocol was used for titer determination of plasma samples from a patient, taken before and after extra-corporal immunoadsorption treatment with Glycosorb-ABO, A-column. Agglutination results with Glycobeads-A are shown in FIG. 7, as well as the results obtained from donor RBC titrations.

Results showed a three titre step reduction of anti-A after Glycosorb A treatment scored with either Glycobeads-A or donor RBCs. The final titer differed, presumably due to different amounts of antigen A on Glycobeads and donor RBCs.

Results showed a three titre step reduction of anti-A after Glycosorb A treatment scored with either Glycobeads-A or donor RBCs. The final titer differed, presumably due to different amounts of antigen A on Glycobeads and donor RBCS.

1-6. (canceled)

7. A method for the analysis of a substance, virus, bacteria, or a cell in a sample, comprising the steps:
purifying the sample by bringing a product comprising at least one carbohydrate or carbohydrate derivative bound to a polymer particle or bead in contact with the sample, said carbohydrate or carbohydrate derivative having binding affinity to said substance, virus, bacteria, or cell, wherein said substance, bacteria, virus, or cell specifically binds to the carbohydrate or carbohydrate derivative of said product;
eluting the substance, bacteria, virus, or cell from the carbohydrate or the carbohydrate derivative of the product;
adding in different dilutions to the eluted substance, bacteria, virus, or cell, said product having binding affinity to the eluted substance, bacteria, virus, or cell;
obtaining a mixture of substance, bacteria, virus, or cell bound to said product;
adding to a gel the mixture of substance, bacteria, virus, or cell bound to said product through which it is allowed to migrate, followed by detection of said substance, bacteria, virus, or cell.

8. The method according to claim 7, wherein the purifying step further comprises partially or completely purifying the sample containing the substance, bacteria, virus, or cell on an affinity gel or in an affinity column.

9. The method according to any one of claim 7, wherein the migration through the gel is promoted by centrifugation, application of a magnetic field, or application of an electric field.

10. The method according to claim 7, wherein the gel is a microtyping card.

11. The method according to claim 7, wherein the carbohydrate or carbohydrate derivative is covalently bound to the polymer particle or bead.

12. The method according to claim 7, wherein the carbohydrates or carbohydrate derivatives have the ability to bind to anti-blood group A or B antibodies.

13. The method according to claim 7, wherein the carbohydrates and carbohydrate derivatives contain the blood group determinants type 1, type 2, type 3 or type 4 of blood group A, B, AB, or O, and di-, tri-, tetra- or higher saccharide parts thereof, the Lewis a, b, x, or y substances, sialic acid containing structures, ganglioside structures or parts thereof, Galili structures, Gal-alpha1-4Gal-, Gal-alpha1-3Gal- containing structures, lactosamine containing structures, GlcNAc-beta1-3Gal, GalNAc-beta1-3Gal- containing structures.

14. The method according to claim 7, wherein the beads are made of polystyrene.

15. The method according to claim 7, wherein the polymer particle or bead is a microsphere or a nanosphere.

16. The method according to claim 7, wherein the product is magnetic.

17. The method according to claim 7, wherein the product used for the purification of the sample is the same as the product added to the eluted substance, bacteria, virus, or cell.

18. The method according to claim 8, wherein the affinity column is a Glycosorb A or B column.

19. The method of claim 7 wherein in the adding to a gel step, the gel is in a column.
